(19) United States
(12) Patent Application Publication
(10) Pub. No.: US 2013/0225905 A1
(43) Pub. Date: Aug. 29, 2013

(54) DEVICE THAT IS IMPLANTABLE IN THE TEMPORAL BONE FOR DELIVERING A MATERIAL, AND HEARING AID PROVIDED WITH SUCH A DEVICE

(71) Applicants: Alexis Bozorg Grayeli, Paris (FR); Olivier Sterkers, Paris (FR); Yann Nguyen, Paris (FR); Mathieu Miroir, Paris (FR); Evelyne Ferrary, Paris (FR)

(72) Inventors: Alexis Bozorg Grayeli, Paris (FR); Olivier Sterkers, Paris (FR); Yann Nguyen, Paris (FR); Mathieu Miroir, Paris (FR); Evelyne Ferrary, Paris (FR)

(73) Assignee: INSERM (INSTITUT NATIONAL DE LA SANTÉ ET DE LA RECHERCHE MÉDICALE), Paris (FR)

(21) Appl. No.: 13/817,465

(22) PCT Filed: Aug. 18, 2011
(86) PCT No.: PCT/FR2011/051930

§ 371 Date: May 17, 2013

(30) Foreign Application Priority Data
Aug. 19, 2010 (FR) 10 56667

(51) Int. Cl. A61F 11/00

(52) U.S. Cl. CPC A61F 11/00
USPC 600/12

(57) ABSTRACT

A device that is implantable in the temporal bone for dispensing at least one component of a liquid product, comprising a catheter and means for moving the component along the catheter, wherein the product incorporates particles for transporting the component, the particles being movable under the effect of a magnetic field, wherein the catheter comprises portions for discharging the component from the catheter, the discharging portions being distributed along the catheter, and wherein the moving means comprise means for generating a magnetic field near each discharging portion so as to move the product from discharging portion to discharging portion, the field-generating means comprising windings that surround the discharging portions and that are connected to a control module designed to successively power at least some of the windings.

A hearing aid, such as a cochlear implant, comprising an internal unit having an electrode holder provided with such a device.

### TECHNICAL FIELD OF THE INVENTION

The present invention relates to a device that is implantable in the temporal bone for introducing a product, such as a medicinal product, into the human body, more specifically into the inner ear. The invention also relates to a hearing aid provided with such a device.

### BACKGROUND OF THE INVENTION

There are medicines available for the treatment of hearing problems, more particularly of problems affecting the inner ear. To be fully effective, these medicines have to be administered directly into the cochlea.

A device that is implantable in the cochlea comprises a catheter having one end connected to a liquid product reservoir and that is intended to be arranged under the skin of the cranium, and an opposite end having a hole for discharging the product. However, the placement of these catheters is very delicate.

Moreover, known hearing aids or cochlear implants comprise an external unit, generally arranged behind the ear, and an internal unit. The external unit comprises an extracranial speech processor for picking up external sounds and converting them into a sequence of electrical signals that are sent to a transmitter in order to be transmitted to an intracranial receiver of the internal unit. The internal unit comprises an electrode holder to which the intracranial receiver is connected in such a way that the cochlear implant can excite the auditory receptors of the cochlea in response to the electrical signals. The electrode holder of the cochlear implant has the form of a thin tube, which is engaged in the cochlea in such a way that the tube extends substantially along the entire length of the curvilinear course of the latter.

It has been envisioned to this end that the cochlear implant can serve to administer the medicines.

A hearing aid has thus been equipped with a catheter that extends along the electrode holder of the cochlear implant. The catheter has one end connected to a liquid product reservoir arranged under the skin of the cranium, and an opposite end having one or more holes for discharging the product. The catheter is combined with means for moving the product in the catheter as far as the discharge hole of the latter.

An object of the invention is to improve the efficacy of the injection of product into the body of a patient and more specifically into the inner ear thereof.

### SUMMARY OF THE INVENTION

To this end, according to the invention, an implantable device is provided for administering at least one component contained in a liquid product, said device comprising a catheter and means for moving the component along the catheter. The product incorporates particles for transporting the component, these particles being movable under the effect of a magnetic field. The catheter comprises portions for discharging the component from the catheter, the discharging portions being distributed along the catheter, and the moving means comprise means for generating a magnetic field near each discharging portion so as to move the component from discharging portion to discharging portion. The means for generating a magnetic field comprise windings that surround the discharging portions and that are connected to a control module designed to successively power at least some of the windings.

The plurality of discharging portions distributed along the catheter, and the means for moving the product arranged to bring the component to each discharging portion, allow the component to escape from the catheter at the locations most suitable for effective diffusion of the component in respect of the desired effect. This embodiment of the means for generating a magnetic field has a simple structure that allows effective movement of the component. In addition, this makes it possible to move the component without moving the liquid product itself, such that the pressure in the catheter and of the liquids of the inner ear is not modified by the migration of the component.

Preferably, the control module is arranged as an intracranial module and, advantageously, the reservoir is connected to the catheter in order to permit subcutaneous implantation of the reservoir.

This avoids the device having a bulky part that is exposed to view.

According to a particular feature, the reservoir and the catheter are arranged in such a way that the catheter extends in proximity to the external auditory meatus of the wearer of the device, and, preferably, the device comprises an auxiliary element for moving the component of the product, this auxiliary element having an elongate outer envelope which is arranged to be introduced into the external auditory meatus and which contains magnetic means for creating at least one magnetic field that is movable along the catheter.

This allows the component of the product to be moved along the catheter more rapidly and more effectively.

Advantageously, the magnetic means of the auxiliary element comprise magnets distributed along a band that goes round two reels in such a way as to extend in a longitudinal direction of the envelope, one of the reels being connected to a rotational driving motor.

This embodiment is particularly simple.

Advantageously, a magnetic shielding screen extends longitudinally in the auxiliary element in order to isolate the magnets on the strips of the band extending on either side of the reels.

The shielding screen makes it possible to isolate the magnets carried by the strip adjacent to the catheter from the magnets carried by the other strip, in such a way as to ensure that the magnetic field generated by the latter does not disturb the movement of the component of the product.

The invention also relates to a hearing aid such as a cochlear implant, comprising an internal unit with an electrode holder along which extends a catheter connected to a liquid product reservoir and combined with means for moving particles which are sensitive to a magnetic field and which are combined with at least one component of the product. The catheter has portions for discharging the component from the catheter, the discharging portions being distributed along the catheter, and the moving means comprise means for generating a magnetic field near each discharging portion so as to move the component from discharging portion to discharging portion. The means for generating a magnetic field comprise windings that surround the discharging portions and that are connected to a control module designed to successively power at least some of the windings.

Other features and advantages of the invention will become clear on reading the following description of a particular and non-limiting embodiment of the invention.

### BRIEF DESCRIPTION OF THE DRAWINGS

Reference will be made to the attached drawings, in which:

FIG. 1 is a partial schematic view of a hearing aid according to the invention, fitted in an ear of a user;

FIG. 2 is an enlarged view, in longitudinal section, of the electrode holder of this hearing aid,

FIG. 3 is a schematic sectional view of an auxiliary element for moving the product.

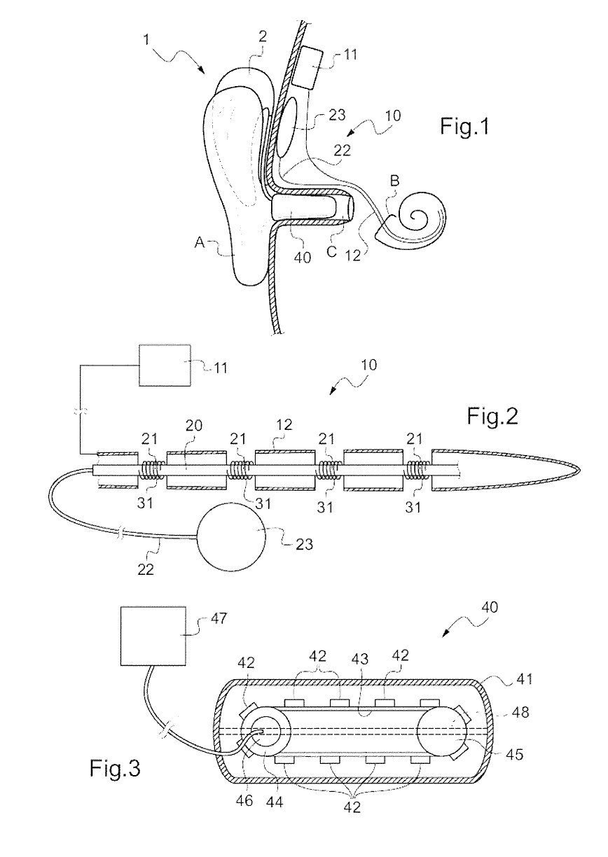

### DETAILED DESCRIPTION

With reference to the figures, the hearing aid according to the invention, which is of the cochlear implant type, comprises an external unit and an internal unit, which are designated generally and respectively as 1 and 10.

The external unit 1 comprises, in a manner known per se, a housing 2 which is generally arranged behind the pinna A of the ear of the user and which contains an extracranial processor for picking up external sounds and converting them into a sequence of electrical signals that are sent to a transmitter connected to the processor. The housing 2 also comprises a power source for supplying power to the processor and the transmitter.

The internal unit 10, the actual cochlear implant, has a control module 11, and an electrode holder 12 separated from the control module 11 and connected to the latter by electrical conductors. The control module 11 comprises a receiver for receiving signals transmitted wirelessly by the transmitter of the external unit 1 and for transmitting corresponding electrical signals to the electrode holder 12 of the cochlear implant, in such a way that the electrode holder 12 can excite the auditory receptors of the cochlea B in response to the electrical signals. This method of operation is conventional and will not be described any further here. The electrode holder 12 has the form of a thin tube, which is engaged in the cochlea B in such a way that the tube extends along substantially the entire length of the curvilinear course of the cochlea B.

The hearing aid is equipped with means for dispensing or administering, into the inner ear, at least one component of a liquid product, such as a medicinal component.

These means comprise a catheter 20 which extends along the electrode holder 12 of the cochlear implant, in this case more specifically inside the latter, and has product discharging portions 21 which are distributed along the catheter 20. The discharging portions 21 can comprise at least one opening formed directly in the wall of the catheter 20 or can be made of a material that is permeable to the product, in particular a porous material forming part of the wall of the catheter 20. The catheter 20 has an end portion 22 connected to a product reservoir 23, which is separated from the cochlear implant in order to extend behind the pinna A under the skin of the user's cranium. The reservoir 23 and the end portion 22 are designed and implanted in such a way that the end portion 22 of the catheter 20 extends in proximity to the external auditory meatus C of the person wearing the hearing aid and parallel to the external auditory meatus C.

The catheter 20 is combined with means for moving the component of the product along the entire length thereof. The moving means are arranged to bring the component selectively to the discharging portions 21.

The moving means comprise means for generating a magnetic field near each discharging portion 21 and for attracting the component of the product thereto, as will be explained below.

The means for generating a magnetic field comprise windings or solenoids 31 which surround the discharging portions 21 and are connected by electrical conductors to the control module 11, which is likewise arranged to successively power at least some of the windings.

The moving means comprise an auxiliary element for moving the product, this auxiliary element having an elongate outer envelope 41 which is designed to be introduced into the external auditory meatus C and which contains magnetic means for creating at least one magnetic field that is movable along the outer envelope 41. The magnetic means of the auxiliary element 40 comprise magnets 42 distributed along a band 43 that goes round two reels 44, 45 in such a way as to extend in a longitudinal direction of the envelope 41. The reel 44 is connected to a rotational driving motor 46 mounted in the body of the element 41 and connected to a power module 47 contained in the envelope 41. The power module 47 incorporates a battery that can be recharged via a connector (not shown) to the electricity supply. The power module 47 can be arranged to periodically control the driving of the motor 46, or on command from the control module 11 to which it is connected by a wireless link. A magnetic shielding screen 48 extends in the envelope 41 in order to isolate the magnets 42 on the strips of the band 43 extending on either side of the reels 44, 45. When the envelope 41 of the auxiliary element 40 is in place in the external auditory meatus, the upper strip of the band 43 extends in proximity to, and parallel to, the end portion 22 of the catheter 20.

The product incorporates particles that are movable under the effect of a magnetic field. The particles have a diameter of between approximately 10 nm and 500 nm and comprise a paramagnetic core covered by a protective layer which carries chemical end groups for attachment of the component, here an active principle. The core is made of one of the following materials: magnetite, maghemite, iron-based metal oxide, steel alloy. The protective layer is made of one of the following materials: a natural polymer such as a carbohydrate; a synthetic organic polymer, such as a polyethylene glycol, a polylactic acid; a silica; gold. The chemical end groups comprise at least one of the following groups: an amine group, a carboxylic group, an aldehyde group, a thiol group. The particles are contained in a solution with a composition identical or close to the liquids of the inner ear, and the discharging portions are designed to permit the passage of the component by passive or active diffusion by the magnetic field.

It will be appreciated that the particles are attracted by the magnetic field produced by the magnets 42 and by the magnetic fields produced selectively by the solenoids.

The rotation of the reel 44 thus causes the magnets 42 of the upper strip to move toward the catheter 20. Since all the magnets are arranged to have the same pole oriented upward when they are on the upper strip of the band 43, said magnets 42 will attract the particles as they move along the upper strip (the magnetic shielding screen 48 ensures that the magnetic field of the magnets 42 does not exert a force of attraction on the particles when the magnets 42 are on the lower strip of the band 43).

The solenoids 31 for their part are controlled independently of one another, so as to attract the particles to the discharging portion around which they extend.

This makes it possible for the product in the proximal and intermediate part of the catheter to be brought initially toward the part inserted in the cochlea and thereafter to the discharging portion from which the product will leave the catheter.

It is also possible to power one or more of the solenoids permanently in order to block the passage of the product.

The auxiliary movement element is intended to be left in the external auditory meatus temporarily, i.e. for a few minutes or tens of minutes, namely the time needed to bring particles in proximity to that portion of the catheter received in the electrode holder 12. This is particularly useful when it is necessary to bring particles rapidly into the cochlea, for example after a recharging of the reservoir 23.

Of course, the invention is not limited to the described embodiment, but instead encompasses any variant covered by the scope of the invention as defined in the claims.

In particular, the product can have a different composition. The particles can serve only to drive the fluid and no longer as a support for the active principles.

The auxiliary element 40 for moving the product is optional. Although its use is particularly advantageous in the described embodiment since it makes it possible to accelerate the transport of the particles to the discharging portions by compensating for the relative slowness of the movement occasioned by the solenoids, the auxiliary element can be used alone or in combination with other types of moving means, such as moving means using an electrical field. The auxiliary element can likewise be used to move a product toward and into a catheter having a single terminal outlet.

The moving means can be arranged to move all of a product (for example by a pumping effect) or only some of it as the sole particles.

Solenoids can extend around the discharging portions, or upstream or downstream of these.

The device implantable in the temporal bone, in accordance with the invention, can be implanted alone in the cochlea, without fitting a cochlear implant, or, by contrast, can be included in a cochlear implant.

The power module 47 can be housed in the external unit.

The hearing aid can comprise a part designed to be placed in one of the following regions of the inner ear: sacculus, vestibule, endolymphatic sac, semicircular canals, cochlea.

1. A device that is implantable in the temporal bone for administering at least one component of a liquid product, comprising a catheter and means for moving the component along the catheter, wherein the product incorporates particles for transporting the component, the particles being movable under the effect of a magnetic field,
wherein the catheter comprises portions for discharging the component from the catheter, the discharging portions being distributed along the catheter, and wherein the moving means comprise means for generating a magnetic field near each discharging portion so as to move the product from discharging portion to discharging portion, the field-generating means comprising windings that surround the discharging portions and that are connected to a control module arranged to successively power at least some of the windings.

2. The device as claimed in claim 1, in which the control module is arranged as an intracranial module.

3. The device as claimed in claim 1, in which the reservoir is connected to the catheter in order to permit subcutaneous implantation of the reservoir.

4. The device as claimed in claim 3, in which the reservoir and the catheter are arranged in such a way that the catheter extends in proximity to the external auditory meatus of the wearer.

5. The device as claimed in claim 4, comprising an auxiliary element for moving the product, this auxiliary element having an elongate outer envelope which is arranged to be introduced into the external auditory meatus and which contains magnetic means for creating at least one magnetic field that is movable along the outer envelope.

6. The device as claimed in claim 5, in which the magnetic means of the auxiliary element comprise magnets distributed along a band that goes round two reels in such a way as to extend in a longitudinal direction of the outer envelope, one of the reels being connected to a rotational driving motor mounted in the outer envelope of the device.

7. The device as claimed in claim 6, in which a magnetic shielding screen extends inside the outer envelope in order to isolate the magnets on the strips of the band extending on either side of the reels.

8. The device as claimed in claim 1, in which the particles comprise a paramagnetic core covered by a protective layer which carries chemical end groups for attachment of an active principle.

9. The device as claimed in claim 8, in which the particles have a diameter of between approximately 10 nm and 500 nm.

10. The device as claimed in claim 1, in which the product comprises a solution with a composition close or identical to the liquids of the inner ear, and the discharging portions are designed to permit the passage of the component by passive or active diffusion.

11. A hearing aid of the cochlear implant type, comprising an internal unit with an electrode holder which is provided with a device for administering at least one liquid product component, wherein the device comprises
a catheter and means for moving the component along the catheter, wherein the product incorporates particles for transporting the component, the particles being movable under the effect of a magnetic field,
wherein the catheter comprises portions for discharging the component from the catheter, the discharging portions being distributed along the catheter, and wherein the moving means comprise means for generating a magnetic field near each discharging portion so as to move the product from discharging portion to discharging portion, the field-generating means comprising windings that surround the discharging portions and that are connected to a control module arranged to successively power at least some of the windings.

12. The hearing aid as claimed in claim 11, which the catheter extends inside the electrode holder.

13. The hearing aid as claimed in claim 11, comprising a part that is designed to be placed in a region of the inner ear selected from the group consisting of: cochlea, sacculus, vestibule, endolymphatic sac, and semicircular canals.
